(19) United States
(12) Patent Application Publication
(10) Pub. No.: US 2025/0160824 A1
(43) Pub. Date: May 22, 2025

(54) APPARATUS AND METHODS FOR JOINING BONES

(71) Applicant: Medline Industries, LP, Northfield, IL (US)

(72) Inventors: Ryan Niver, Glenview, IL (US); Michael Ksenich, Northfield, IL (US); Dinesh Koka, Winter Park, FL (US)

(21) Appl. No.: 19/029,636

(22) Filed: Jan. 17, 2025

Related U.S. Application Data
(62) Division of application No. 17/407,182, filed on Aug. 19, 2021, now Pat. No. 12,220,123.

(51) Int. Cl. A61B 17/064; A61B 17/16; A61B 17/17

(52) U.S. Cl. CPC A61B 17/0642; A61B 17/1615; A61B 17/1728; A61B 2017/0641; A61B 2017/0648; A61B 17/1682; A61B 17/1714; A61B 17/1739; A61B 17/1775; A61B 17/1782

(57) ABSTRACT

Apparatus and methods are described for use in securing a first bone relative to a second bone using a pair of surgical staples or, alternatively, a surgical staple and a bone screw. When a pair of staples are used, one of the legs of one of the staples may pass between the legs of the another of the staples. When a staple and a bone screw are used, the bone screw may pass between the legs of the staple. A guide tool can be used during the drilling of the holes for the legs of the staples or the legs of the staple and a bone screw. The guide tool provides guides for drilling guide holes for the staples or for the screw and staple. The guides can be fixed relative to each other during drilling of the guide holes.

### CROSS-REFERENCE TO RELATED APPLICATION

This application is a divisional of U.S. application Ser. No. 17/407,182 filed Aug. 19, 2021, which is hereby incorporated by reference in its entirety.

### FIELD

Apparatus and methods for joining or fusing two bones together are described herein and, more specifically, apparatus and methods for drilling guide holes in bones for receiving surgical staples or a surgical staple and a bone screw.

### BACKGROUND

Screws are commonly used for joining or fusing two bones or bone pieces together. The screw or screws may cross a joint, fracture or osteotomy. For example, screws can be used to fuse metatarsal phalangeal (MTP) joints to relieve pain. By way of another example, screws can be used in a Lapidus procedure to fuse the joint between the first metatarsal bone and the medial cuneiform. Instead of screws, fusions can be made using compression staples. A problem with using screws or staples, particularly where multiple such fasteners are used in a single procedure, is that often guide holes for receiving the screws or legs of the staples are drilled free-hand. Although suitable fusions can be made using holes that are drilled free-hand, this can be time consuming and potentially prone to errors in the relative positions and orientations of the staple legs or screw.

The guide tools and methods described herein can address these problems.

### BRIEF DESCRIPTION OF THE DRAWINGS

FIG. 1 is a perspective view of an embodiment of a guide tool for use in drilling holes in bones for insertion of a first surgical staple and a second surgical staple, the guide tool having a set of rearward guides for use in drilling holes to receive legs of the first surgical staple and a moveable set of forward guides for use in drilling holes to receive legs of the second surgical staple;

FIG. 2 is a top plan view of the guide tool of FIG. 1;

FIG. 3 is a left side elevation view of the guide tool of FIG. 1;

FIG. 4 is a right side elevation view of the guide tool of FIG. 1;

FIG. 5 is a front side elevation view of the guide tool of FIG. 1;

FIG. 6 is a rear perspective view of a movable bracket of the forward guides of the guide tool of FIG. 1;

FIG. 7 is a detailed view of the front end of the handle of FIG. 1;

FIG. 8 is an exploded view of the guide tool of FIG. 1;

FIG. 9 is a perspective view of an operative portion of the guide tool of FIG. 1 positioned adjacent a bone in after guide holes have been drilled in the bone and staples inserted into the guide holes;

FIG. 10 is another perspective view of an operative portion of the guide tool of FIG. 1 positioned adjacent a bone in before guide holes have been drilled in the bone and before staples inserted into the guide holes;

FIG. 11 is perspective view, similar to that of FIG. 10, of an operative portion of the guide tool of FIG. 1 positioned adjacent a bone in after some but not all of the guide holes have been drilled in the bone and before staples are inserted into the guide holes;

FIG. 12 is a perspective view of a pair of staples having been inserted into holes drilled in the bones;

FIG. 13 is a perspective view of a second embodiment of a guide tool for use in drilling holes in bones for insertion of a surgical staple and a bone screw, the guide tool having a set of rearward guides for use in drilling holes to receive legs of the surgical staple and a moveable forward guide for use in drilling a hole to receive the bone screw, the guide tool being positioned adjacent bones for drilling holes;

FIG. 14 is a different perspective view of the tool of FIG. 13 positioned adjacent bones for drilling holes, showing a guide wire inserted through a pin disposed in the forward guide;

FIG. 15 is a perspective view of a staple and a screw having been inserted into holes drilled in the bones;

FIG. 16 is a perspective view of an alternative embodiment of a guide tool for use in drilling holes in bones for insertion of a first surgical staple and a second surgical staple, the guide tool having a set of rearward guides for use in drilling holes to receive legs of the first surgical staple and a moveable set of forward guides for use in drilling holes to receive legs of the second surgical staple, and having multiple openings through which guide pins can be inserted and passed into adjacent bones for positing the tool prior to the drilling of some or all of the guide holes;

FIG. 17 is a perspective view of the tool of FIG. 16, showing guide holes having been drilled for the legs of the staples;

FIG. 18 is another alternative tool, having a portion that can clamp or at least partially anchor onto an already-inserted staple, and a forward portion with a pair of drilling guides;

FIG. 19 is another alternative tool, having a portion that can clamp or at least partially anchor onto an already-inserted staple, and a forward portion with an offset drilling guide for drilling a guide hole for a screw;

FIG. 20 is a perspective view of a surgical staple having two legs;

FIG. 21 is a side elevation view of the surgical staple of FIG. 20, showing legs of the staple at acute angles relative to a bridge;

FIG. 22 is a perspective view of a surgical staple having four legs;

FIG. 23 is a side elevation view of the surgical staple of FIG. 22, showing legs of the staple at acute angle relative to a bridge;

FIG. 24 is a perspective view of a surgical staple having four legs; and

FIG. 25 is a side elevation view of the surgical staple of FIG. 22, showing legs of the staple at acute angle relative to a bridge.

### DETAILED DESCRIPTION

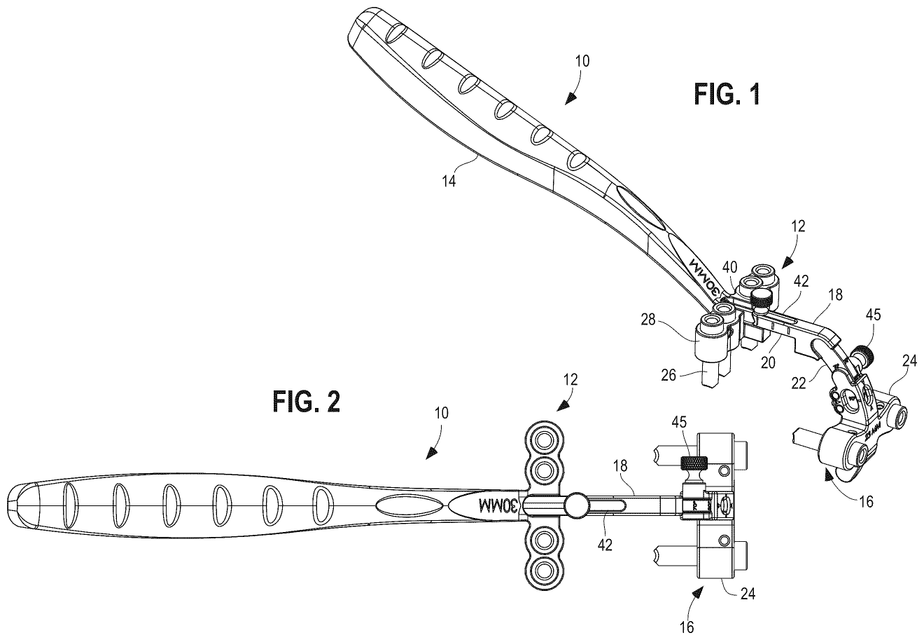

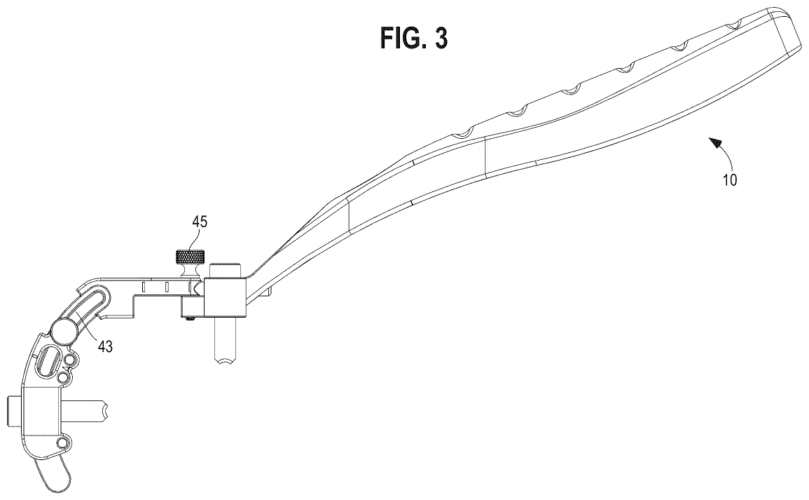

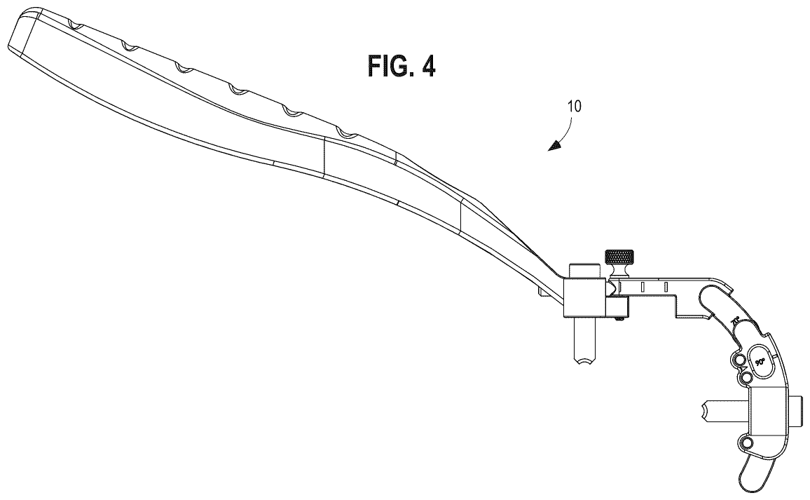

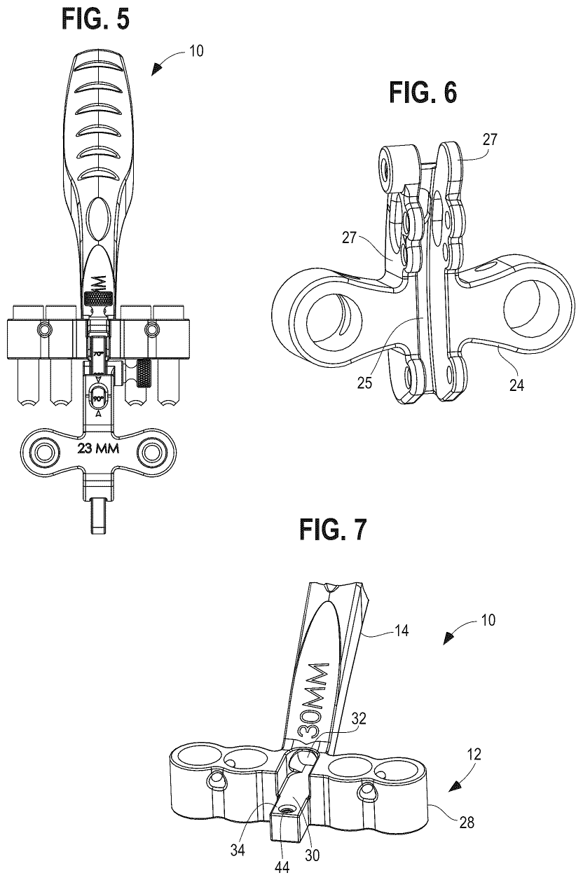

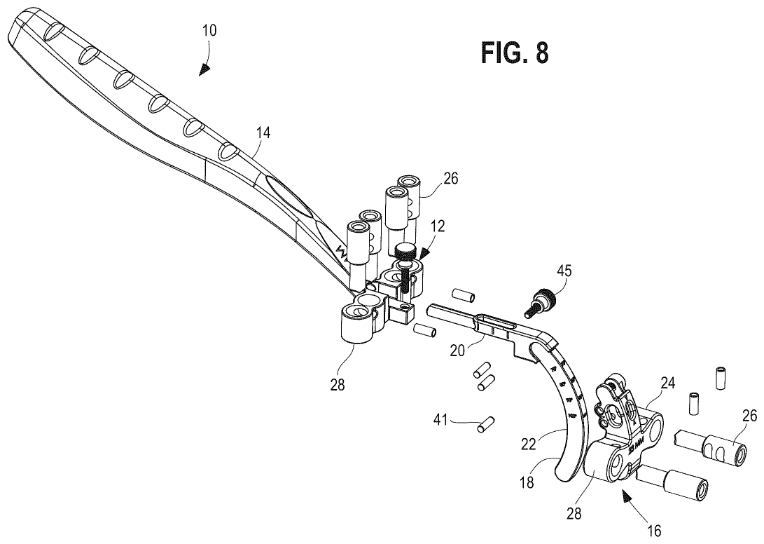

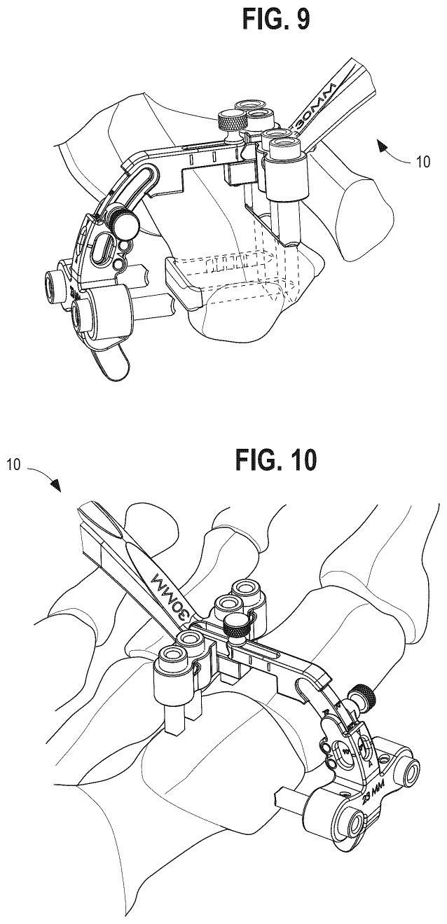

As described herein and shown in FIGS. 1-25, apparatus and methods are disclosed for use in securing a first bone relative to a second bone using a pair of surgical staples or, alternatively, a surgical staple and a bone screw. When a pair of staples are used, advantageously at least one of the legs of one of the staples passes between the legs of the another of the staples. If the legs of the staple are coplanar, then this can beneficially result in intersecting planes of securement. When a staple and a bone screw are used, advantageously the bone screw passes between the legs of the staple. This can beneficially result in the bone screw intersecting a plane of the legs of the staple. A guide tool can be used during the drilling of the holes for the legs of the staples or the legs of the staple and a bone screw. Advantageously, the guide tool provides guides for drilling the guide holes such that the guides can be fixed relative to each other to improve accuracy during drilling and make the fusion procedure faster, simpler and readily reproducible.

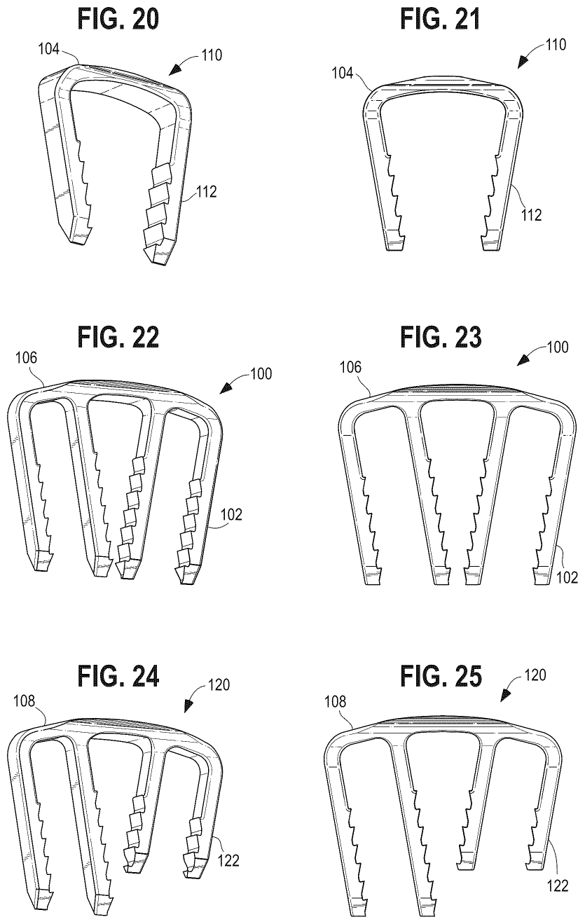

A first embodiment of the guide tool 10 includes a rearward set of drill guides 12 fixed relative to a handle 14 and an adjustable forward set of drill guides 16 that can be selectively fixed relative to the rearward set of drill guides 12, aspects of which are shown in FIGS. 1-12. The guide tool 10 can be used for drilling guide holes in bones for insertion of a legs 102 of a first surgical staple 100 and legs 112 of a second surgical staple 110. The rearward drill guides 12 can be used to drill a plurality of parallel guide holes for receiving the legs 102 of the first staple 100 which, in the exemplary embedment, has four legs 102 generally in a first staple plane, such as those shown in FIGS. 22-25. The forward drill guides 16 can be used to drill a plurality of parallel holes for receiving the legs of the second staple which, in the exemplary embodiment, has two legs generally in a second staple plane, such as shown in FIGS. 20 and 21. The guide tool 10 is configured such that the holes are aligned so that the first and second staple planes intersect once the staples are inserted into the bones, as shown in FIG. 12.

Broadly, the guide tool 10 includes the handle 14 with an attached rearward set of drill guides 12. An adjustable arm 18 has a linear segment 20 that can be selectively attached to the handle 14 in a variety of different positions. The adjustable arm 18 also has an arcuate segment 22 that is spaced from the handle by the linear segment of the arm. A bracket 24 is slidable along the arcuate segment 22 and carries the forward set of drill guides 16. The bracket, and thus the forward set of drill guides, can be selectively fixed relative to the arcuate segment of the arm.

Each of the drill guides 12, 16 includes a cylindrical sleeve 26 having a through-opening extending along a central axis thereof. In use, a drill bit can be inserted into the through-opening and used to drill a hole. The through-opening and drill bit are preferably sized that play between the drill bit and the sleeve is minimized so that a hole can be drilled with accuracy. The drill guides also include a separate holder 28 for each of the cylindrical sleeves. Each of the holders includes a through-bore for receiving part of one of the cylindrical sleeves. Each of the cylindrical sleeves can optionally have a serrated edge at the distal tip thereof for seating on an adjacent bone.

The cylindrical sleeves are axially and rotationally secured in the through-bores of the holders. Each of the holders has a pair of aligned apertures tending perpendicular relative to a longitudinal axis passing through a center of the holder. An inner circumferential wall of the holder has partially dished in alignment with the aligned apertures. Each of the cylindrical sleeves also has a dished portion on an outer circumference thereof, extending generally perpendicular relative to the central axis thereof. When one of the cylindrical sleeves is received with the through-bore of one of the holders, the dished portion of the cylindrical sleeve is positioned to face the dished portion of the inner circumferential wall of the holder such that an opening of circular cross-section is formed therebetween. When so arranged, a pin can be positioned with each end being held by the aligned apertures and through the opening formed by the dished portions. The pin is thus fixed to the holder via the aligned apertures thereof. Engagement between the fixed pin and the dished portion of the cylindrical sleeve both prevents rotation of the cylindrical sleeve within the holder and axial movement of the cylindrical sleeve within the holder.

The handle 14 includes a longitudinally extending grasping portion, which can optionally be ergonomically contoured for being held by the hand of a surgeon or other user. As mentioned above, the rearward set of drill guides 12 are attached to the handle 14. More specifically, there are four drill guide holders integrally formed at end of the handle. The four holders are arranged in a line that is generally perpendicular to the grasping portion of the handle, as shown in FIG. 2. Other arrangements of the holders relative to the handle can be made, e.g., parallel or angled; and the holders do not have to be in a line, but can be in other non-linear arrangements. In the illustrated embodiment, there are four holders. It will be understood that a different number of holders can be used depending upon the number of staple legs to be inserted into holes, e.g., two, three, etc.

The handle includes an open channel 30 formed between the four holders, as seen in FIG. 7. The channel is configured to receive the linear segment of the adjustable arm. More specifically, two of the holders are on one side of the channel and two of the holders on another side of the channel. The channel has a flat bottom wall, a rear end with an obround opening 32, and a projecting end 34 extending forward of the holders. The linear segment of the adjustable arm terminates in a protuberance that is obround in cross-section and keyed to fit within the oval-shaped opening of the rear end of the channel of the handle. A flattened bottom of the protuberance abuts the flat bottom wall of the channel. Accordingly, engagement between the channel and the protuberance of the linear segment of the arm restricts rotation of the arm relative to the handle.

The arm can be selectively attached relative to the handle, as mentioned above. To that end, a threaded screw or knob 40 can be used to secure the arm to the handle. More specifically, the linear segment of the adjustable arm has an elongated slot 42 formed therein. When the protuberance of the linear segment of the arm is received within the channel of the handle, the slot is positioned above a threaded bore 44 formed in the projecting end of the channel. The knob has a threaded shaft depending from a head with a knurled periphery. The threaded shaft of the knob is inserted through the slot of the linear segment of the arm and into the threaded bore. Tightening of the knob can clamp the arm to the handle. The knob can be removed to either detach the arm from the handle or to loosened to allow the arm to be slidingly adjusted relative to the handle and then tightened.

The bracket 24, which carries the forward set of drill guides, is slidable along the arcuate segment. A pair of the holders are provided on the bracket, one on each side of the arcuate bottom wall thereof. The arcuate segment has an arcuate, radially outward surface and an arcuate, radially inward surface. The arcuate segment has a generally rectangular cross-section. The bracket includes an arcuate bottom wall 25, as shown in FIG. 6, with a pair of lateral, upstanding side walls 27. A radius of curvature of the bottom wall of the bracket generally matches a radius of curvature of the arcuate segment of the arm such that the bottom wall of the bracket can slide along the outward surface of the arcuate segment of the arm. Each of the pair of sidewalls has three apertures. The apertures of one of the pair of sidewalls is aligned with the apertures of the other of the pair of sidewalls. Pins 41 are inserted through aligned apertures, such that there are three pins. The apertures, and thus the pins, are aligned such the dowels can slide along the inward surface of the arcuate segment of the arm. The pins and the arcuate bottom wall sandwich the radially inward surface and the radially outward surface of the arcuate segment, respectively, therebetween.

The pair of sidewalls and the bottom walls optionally have windows formed therein through which indicia on the arcuate segment of the arc can be visible to help with positioning the bracket along the arcuate segment of the arm.

The bracket, and thus the forward set of drill guides, can be selectively fixed relative to the arcuate segment of the arm, as mentioned above. The arcuate segment of the adjustable arm has a generally rectangular cross-section. One side of the arcuate segment has a curved, obround-shaped groove 43. One of the sidewalls of the bracket has an extending ear with a threaded through-bore. A knob 45 is provided to secure the bracket on the arcuate segment of the arm. More specifically, the knob has a threaded shaft depending from a head with a knurled periphery. The threaded shaft of the knob is threaded into the threaded through-bore of the ear of the sidewall of the arcuate segment of the arm and into engagement with a bottom of the groove on the arcuate segment of the arm. Tightening of the knob can clamp the bracket to the arm. The knob can be removed to allow the bracket to be slid off the arm or loosened to allow the bracket to be slidingly adjusted relative to the arm and then tightened to fix the bracket in position relative to the rearward set of drill guides. Ends of the groove on the arcuate segment of the arm can limit the extent to which the backet can slide on the arm when then threaded shaft is received within the groove but not engaged with the bottom wall thereof.

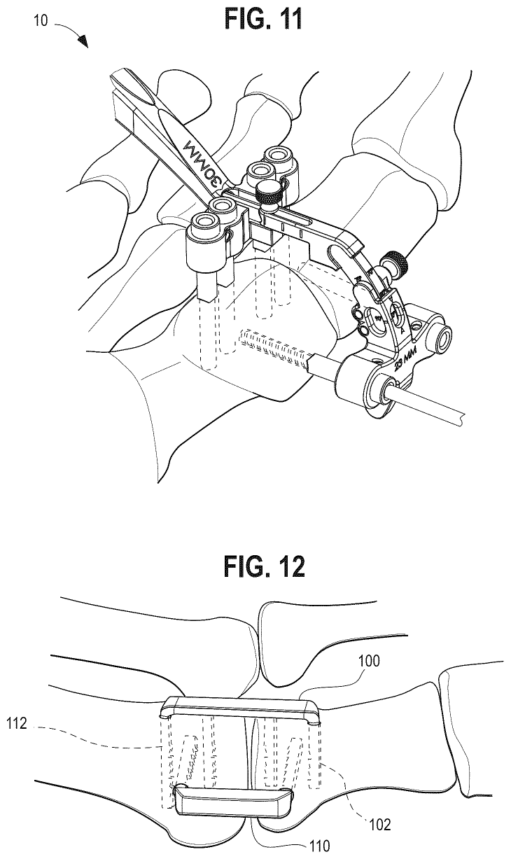

Turning now to a method of using the first embodiment of the guide tool, the handle can be used to position the rearward set of drill guides against a pair of adjacent bones, as shown in FIG. 10. The forward set of drill guides can also be positioned against the pair of adjacent bones, also as shown in FIG. 10, with adjustment to the position of the forward set of drill guides being made using the arm and/or the bracket. As used herein, adjacent bones can also include bone pieces of the same bone that require fusion. Optionally, a guide wire for a cannulated screw can be installed in one of the bones. When used, the guide wire can then be threaded through one of the rearward set of drill guides to help with positioning the guide tool adjacent the bones. A first of the holes can then be drilled using a drill bit—attached to a drill—inserted through the first of the holes. A surgeon can drill the remainder of the holes or, optionally, insert one or more removable pins temporarily through the drill guides to temporarily secure the guide tool to the bone or bones. For example, a hole can be drilled into one of the pair of bones using one of the rearward set of drill guides to guide the drill bit; and another hole can be drilled into the same or a different one of the pair of bones using the forward set of drill guides to guide the drill bit, as shown in FIG. 11. Removable pins can be inserted into those holes to temporarily secure the guide tool to the bone or bones. The remainder of the holes can be drilled, after which the pins can be removed and the guide tool removed from adjacent the bones. Staples can then be inserted into the holes that are drilled into the bones to fuse the bones together, as shown in FIG. 12.

Preferably, though not necessarily, the legs of each of the staples in their resting or unbiased state are at acute angles relative to a bridge that joins the legs, as shown in FIGS. 20-25. The staples can optionally be made of a shape memory metal, such as nitinol. The drilled holes made using the rearward set of drill guides are perpendicular to each other, as are the drilled holes made using the forward set of drill guides. The legs of the staples can be temporarily bent to be generally perpendicular relative to the bridges and generally parallel relative to each other and, when in that arrangement, inserted into the holes. An insertion tool, such as that disclosed in U.S. patent application Ser. No. 17/322,580, filed May 17, 2021, which is hereby incorporated herein by reference in its entirety, can engage with the staple to bend and temporarily hold the legs in the perpendicular and parallel arrangement for insertion into the holes. Once the legs are almost completely inserted, the insertion tool can be disengaged from the staple and the staple legs inserted the rest of the way into the holes. Advantageously, the shape memory properties of the staple can cause the legs to want to return to their acutely angled orientation relative to the bridge, thereby compressing the adjacent bones together, preferably with a compressing force that is greater than if the legs in their resting or neutral state were generally perpendicular relative to the bridge.

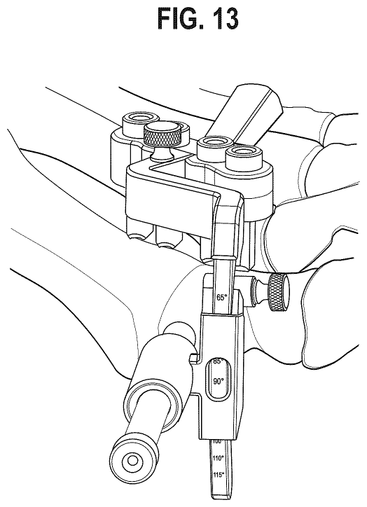

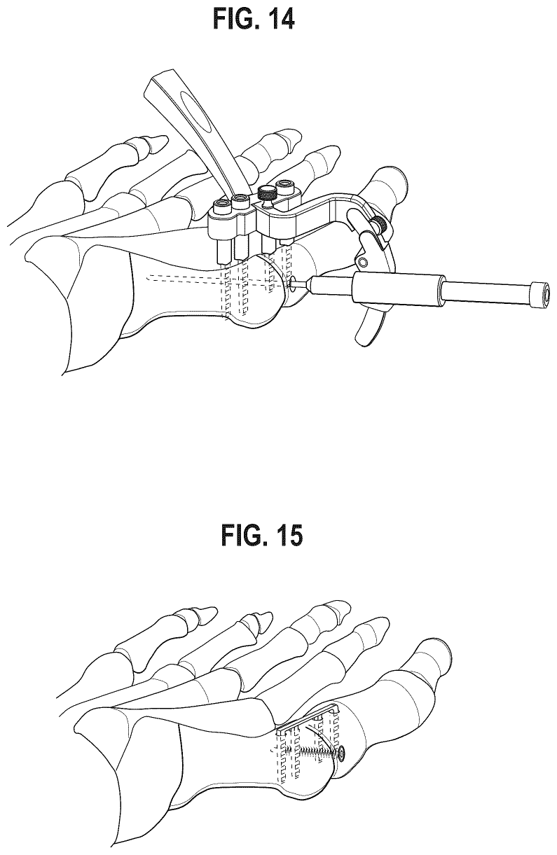

A second embodiment of the guide tool is shown in use in FIGS. 13 and 14, with like parts having like numbers as compared with the first embodiment of the guide tool. The second embodiment of the guide tool differs from that of the first embodiment in that instead of being used to drill holes for a first staple and a second staple, the guide tool is configured to drill holes for only the legs of one staple and another hole for a bone screw. In particular, the forward set of drill guides is replaced by a single drill guide. Because a bone screw is used instead of one of the staples, the bone screw must be inserted such that it spans between both bones; thus, the hole drilled for the bone screw must also span between both bones. This is accomplished by the length of the bone screw as well as the angle in which the bone screw is inserted. The forward drill guide is angled such that the drill bit drills a hole that is angled, i.e., not perpendicular, to a plane of the holes for the legs of the staple. The linear and arcuate segments of the adjustable arm are at an obtuse angle relative to each other, as shown in FIGS. 13 and 14, such that a hole drilled using the forward drill guide will is angled to a plate of the holes for the legs of the staple. Like the first embodiment, a guide wire can optionally be used for aligned the guide tool of the second embodiment with the bone or bones. The holes can be sequentially drilled using the drill guides and, optionally, one or more pins can be used for temporarily securing the guide tool to the bone or bones. After drilling of the guide holes, the staple and the screw can be inserted, as shown in FIG. 15.

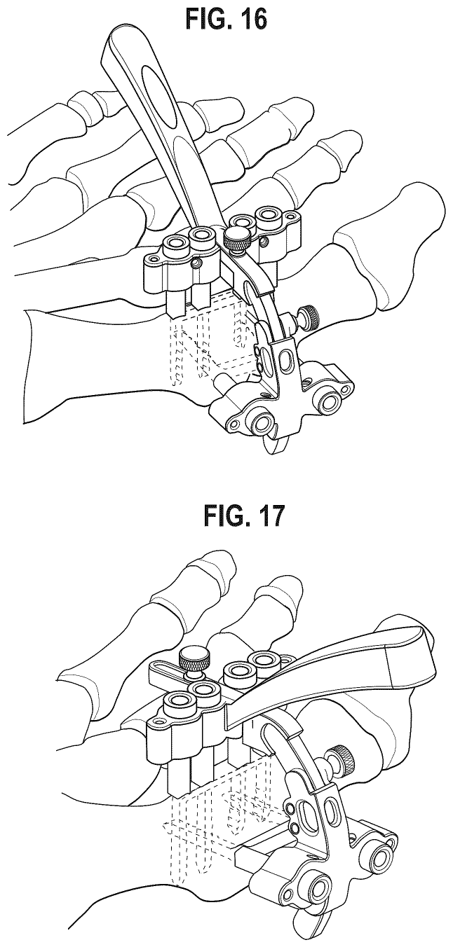

FIGS. 16 and 17 depict an alternative embodiment of a guide tool for use in drilling holes in bones for insertion of a first surgical staple and a second surgical staple, the guide tool having a set of rearward guides for use in drilling holes to receive legs of the first surgical staple and a moveable set of forward guides for use in drilling holes to receive legs of the second surgical staple. This embodiment differs from the first embodiment in that the handle is of a different size, the arm lacks the extension so that it can be inserted through a rear hole in the channel so that the handle is reversible, and there are multiple openings adjacent the drill guides through which guide pins can be inserted and passed into adjacent bones for positing the tool prior to the drilling of some or all of the guide holes. Any of these features can be incorporated into the other embodiments described herein. FIG. 17 shows guide holes having been drilled for the legs of the staples.

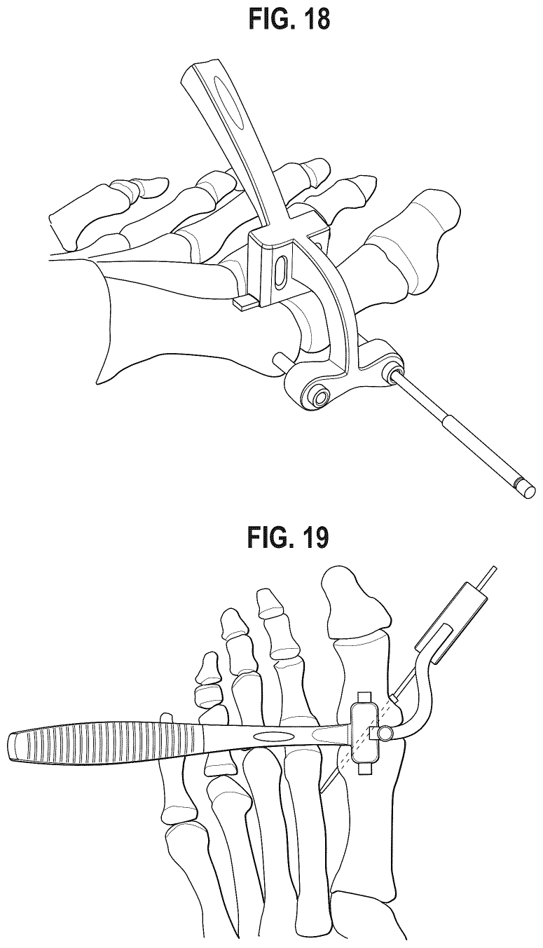

FIGS. 18 and 19 depict addition alternative tools. Each of these two tools has a portion that can clamp or at least partially anchor onto an already-inserted staple, such as groove or slot for receiving the bridge of the staple. Each of these two tools also includes a forward portion with a pair of drilling guides (FIG. 18) for drilling guide holes for a staple, in the illustrated embodiment, a two-legged staple, or an offset drilling guide (FIG. 19) for drilling a guide hole for a screw.

FIGS. 20-25 disclose various embodiments of staples 100, 110, 120, some staples 110 with two legs 112 (FIGS. 20 and 21), some staples 100, 120 with four legs 102, 122 (FIGS. 22 and 23), and one 120 with four legs 122, two of which are shorter than the other two legs (FIGS. 24 and 25). The legs of the staples are connected via a bridge 104, 106, 108, and the legs can optionally have teeth such as those shown. The use of a staple with two legs shorter than another two legs can beneficially be used for various procedures, such as when one of the bones or the operative portion of the bone has a thickness that may not warrant or be suitable for a longer leg. Other shapes and sizes of staples can be used, such as those disclosed in U.S. Patent Appl. Nos. 29/804,413 and 29/804,411, each filed May 17, 2021, which are each hereby incorporated herein by reference in their entireties.

The staples are preferably, though not necessarily, made of nitinol or another shape memory material. Sleeves and arm can be made of surgical grade stainless steel, among other materials. The handle and bracket can also be made of surgical grade stainless steel, suitable thermoplastics, e.g., polyphenylsulfone (PPSU), or outer suitable materials.

All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A method of securing a first bone to a second bone using a first surgical staple having four legs and a second surgical staple having two legs, the method comprising:
drilling a first set of holes in the first and second bones, the first set of holes comprising four holes for receiving the legs of the first surgical staple, the four holes comprising a first pair of holes and a second pair of holes, at least one of the first set of holes being in the first bone and at least one of the first set of holes being in the second bone;
drilling a second set of holes in the first and second bones, the second set of holes comprising two holes for receiving the legs of the second surgical staple, one of the second set of holes being in the first bone and one of the second set of holes being in the second bone, wherein one of the two holes passes between the first pair of holes and another of the two holes passes between the second pair of holes;
inserting the four legs of the first surgical staple in the four holes; and
inserting the two legs of the second surgical staple in the two holes.

2. The method of claim 1, further comprising:
using a first set of drill guides during drilling the first set of holes; and
using a second set of drill guides during drilling the second set of holes;
wherein the first and second set of drill guides are fixed relative to each other during the steps of drilling the first set of holes and drilling the second set of holes.

3. The method of claim 2, further comprising using a guide tool during the steps of drilling the first set of holes and drilling the second set of holes, the guide tool comprising:
a handle having one of the first and second set of drill guides fixed relative thereto;
an adjustable arm selectively fixable relative to the handle and moveable toward and away from the one of the first and second set of drill guides, the adjustable arm having an arcuate segment with another of the first and second set of drill guides selectively fixable relative thereto.

4. A method of securing a first bone to a second bone using a surgical staple having at least two legs and a bone screw, the method comprising:
drilling staple leg holes in the first and second bones for receiving the legs of the surgical staple, at least one of the staple leg holes being in the first bone and at least one of the staple leg holes being in the second bone;
drilling a bone screw hole in both of the first and second bones for receiving the bone screw, wherein the bone screw hole passes between the staple leg holes;
inserting the legs of the surgical staple in the staple leg holes; and
inserting the bone screw in the bone screw hole.

5. The method of claim 4, further comprising:
using a set of staple leg drill guides during drilling the staple leg holes; and
using a bone screw drill guide during drilling the bone screw hole;
wherein staple leg drill guides and the bone screw drill guide are fixed relative to each other during the steps of drilling the staple leg holes and drilling the bone screw hole.

6. The method of claim 5, further comprising using a guide tool during the steps of drilling the staple leg holes and drilling the bone screw hole, the guide tool comprising:
a handle having one of the staple drill guides and the bone screw guide fixed relative thereto;
an adjustable arm selectively fixable relative to the handle and moveable toward and away from the one of the staple drill guides and the bone screw guide, the adjustable arm having an arcuate segment with another of the staple drill guides and the bone screw guide selectively fixable relative thereto.
